(19) United States
(12) Patent Application Publication
(10) Pub. No.: US 2022/0220090 A1
(43) Pub. Date: Jul. 14, 2022

(54) IMPROVED METHODS FOR CONVERTING CANNABIDIOL INTO DELTA9-TETRAHYDROCANNABINOL UNDER NEAT OR APROTIC REACTION CONDITIONS

(71) Applicant: Canopy Growth Corporation, Smiths Falls (CA)

(72) Inventors: Christopher ADAIR, Smiths Falls (CA); Ben GEILING, Smiths Falls (CA); Mohammadmehdi HAGHDOOST MANJILI, Smiths Falls (CA)

(21) Appl. No.: 17/596,347

(22) PCT Filed: Jun. 11, 2020
(86) PCT No.: PCT/CA2020/050807

§ 371 Date: Dec. 8, 2021

Related U.S. Application Data
(60) Provisional application No. 62/860,130, filed on Jun. 11, 2019.

(51) Int. Cl. C07D 311/80; B01J 29/40

(52) U.S. Cl. CPC C07D 311/80; B01J 29/40

(57) ABSTRACT

Disclosed herein is a method for converting cannabidiol (CBD) into a composition comprising Δ⁹-tetrahydrocannabinol (Δ⁹-THC) and Δ⁸-tetrahydrocannabinol (Δ⁸-THC) in which the composition has a Δ⁹-THC:Δ⁸-THC ratio of greater than 1.0:1.0. The method comprises contacting the CBD with a Lewis-acidic heterogeneous reagent under reaction conditions comprising: (i) an aprotic-solvent system; (ii) a reaction temperature that is less than a threshold reaction temperature for the Lewis-acidic heterogeneous reagent and the aprotic-solvent system; and (iii) a reaction time that is less than a threshold reaction time for the Lewis-acidic heterogeneous reagent, the aprotic-solvent system, and the reaction temperature. Methods for converting CBD into a composition comprising Δ⁹-THC and Δ⁸-THC in which the composition has a Δ⁹-THC:Δ⁸-THC ratio of greater than 1.0:1.0 under neat reaction conditions are also provided.

### CROSS-REFERENCE TO RELATED APPLICATION

This application claims priority to and benefit of U.S. Provisional Patent Application Ser. No. 62/860,130 filed on Jun. 11, 2019, which is hereby incorporated by reference.

### TECHNICAL FIELD

The present disclosure generally relates to methods for isomerizing cannabinoids. In particular, the present disclosure relates to methods for converting cannabidiol into primarily Δ⁹-tetrahydrocannabinol and/or mixtures of Δ⁹-tetrahydrocannabinol and Δ⁸-tetrahydrocannabinol.

### BACKGROUND

Since the discovery of specific receptors for cannabinoids in mammalian brain and peripheral tissues, cannabinoids have attracted renewed interest for medicinal and recreational applications. Tetrahydrocannabinol-type (THC-type) cannabinoids are particularly interesting in this respect given their potential psychoactivity. Interestingly, pharmacological studies indicate that some THC-type cannabinoids show similar cannabinoid-receptor-binding affinities but very different psychoactive effects. For example, Δ⁹-tetrahydrocannabinol (Δ⁹-THC) and Δ⁸-tetrahydrocannabinol (Δ⁸-THC) have similar cannabinoid-receptor-binding affinities, yet Δ⁹-THC is reported to be approximately 50% more potent than Δ⁸-THC in terms of psychoactivity. Accordingly, methods for preparing Δ⁹-THC are attractive, as are methods for preparing mixtures of Δ⁹-THC and Δ⁸-THC in which Δ⁹-THC is the major product.

Δ⁹-THC and Δ⁸-THC can both be prepared from cannabidiol (CBD). However, known methods for converting CBD to Δ⁹-THC and/or Δ⁸-THC typically employ chemicals that are dangerous, and/or toxic. Moreover, such methods typically rely on protocols that are generally considered hazardous and/or not suitable for industrial scale reactions (e.g. reagent-addition, quenching, and/or work-up steps that are highly exothermic). Several known methods for converting CBD to Δ⁹-THC and/or Δ⁸-THC also require special care to eliminate oxygen and moisture from the reaction vessel for optimal reactivity and safety. Accordingly, improved methods of converting CBD into Δ⁹-THC and/or Δ⁸-THC are desirable.

### SUMMARY

The present disclosure provides improved methods of converting cannabidiol (CBD) into primarily Δ⁹-tetrahydrocannabinol (Δ⁹-THC) or mixtures of Δ⁹-THC and Δ⁸-tetrahydrocannabinol (Δ⁸-THC) having Δ⁹-THC:Δ⁸-THC ratios of greater than 1.0:1.0. The methods of the present disclosure are suitable for use at industrial scale in that they do not require: (i) complicated and/or dangerous reagent-addition, quenching, and/or work-up steps; and (ii) dangerous and/or toxic solvents and/or reagents. Importantly, the methods of the present disclosure provide access to compositions with ranging Δ⁹-THC:Δ⁸-THC ratios as evidenced by examples disclosed herein. Because the Δ⁹-THC:Δ⁸-THC ratios disclosed herein can be correlated to particular reaction conditions and reagents, the methods of the present disclosure may be tuned towards particular Δ⁹-THC/Δ⁸-THC selectivity outcomes.

Without being bound to any particular theory, the present disclosure reports that the ability to convert CBD into primarily Δ⁹-THC and/or compositions of various Δ⁹-THC:Δ⁸-THC ratios greater than 1.0:1.0 as demonstrated herein is associated with the utilization of Lewis-acidic heterogeneous reagents under aprotic or neat reaction conditions in which reaction temperature and reaction time parameters are carefully selected and controlled. In particular, the examples of the present disclosure indicate that mild reaction temperatures and/or short reaction times favor the formation of Δ⁹-THC over Δ⁸-THC when a Lewis-acidic heterogeneous reagent is utilized in the presence of an aprotic-solvent system. The examples disclosed herein also indicate that the application of Lewis-acidic heterogeneous reagents to the conversion of CBD into primarily Δ⁹-THC or mixtures of Δ⁹-THC and Δ⁸-THC having Δ⁹-THC:Δ⁸-THC ratios greater than 1.0:1.0 is compatible with solvent-free or aprotic-solvent systems provided the reaction temperature and the reaction time are carefully selected and controlled. The utilization of Lewis-acidic heterogeneous reagents under neat reaction conditions or in the presence of particular aprotic solvents for such transformations may obviate the need for the dangerous and/or hazardous solvents that are typical of the prior art. The utilization of Lewis-acidic heterogeneous reagents under neat reaction conditions or in the presence of particular aprotic solvents may also allow product mixtures that are suitable for isolation by simple solid/liquid separations (e.g. filtration and/or decantation). As such, the combination of Lewis-acidic heterogeneous reagents and aprotic or neat reaction conditions appears to underlie one more of the advantages of the present disclosure.

In select embodiments, the present disclosure relates to a method for converting CBD into a composition comprising Δ⁹-THC and Δ⁸-THC, in which the composition has a Δ⁹-THC:Δ⁸-THC ratio that is greater than 1.0:1.0. In such embodiments, the method may comprise contacting the CBD with a Lewis-acidic heterogeneous reagent under reaction conditions comprising: (i) an aprotic-solvent system; (ii) a reaction temperature that is less than a threshold reaction temperature for the Lewis-acidic heterogeneous reagent and the aprotic-solvent system; and (iii) a reaction time that is less than a threshold reaction time for the Lewis-acidic heterogeneous reagent, the aprotic-solvent system, and the reaction temperature.

In select embodiments, the present disclosure relates to a method for converting CBD into primarily Δ⁹-THC. In such embodiments, the method may comprise contacting the CBD with a Lewis-acidic heterogeneous reagent under reaction conditions comprising: (i) an aprotic-solvent system; (ii) a reaction temperature that is less than a threshold reaction temperature for the Lewis-acidic heterogeneous reagent and the aprotic-solvent system; and (iii) a reaction time that is less than a threshold reaction time for the Lewis-acidic heterogeneous reagent, the aprotic-solvent system, and the reaction temperature.

In select embodiments, the present disclosure relates to a method for converting CBD into a composition comprising Δ⁹-THC and Δ⁸-THC, in which the composition has a Δ⁹-THC:Δ⁸-THC ratio that is greater than 1.0:1.0. In such embodiments, the method may comprise contacting the CBD with a Lewis-acidic heterogeneous reagent under neat reaction conditions comprising: (i) a reaction temperature that is less than a threshold reaction temperature for the Lewis-acidic heterogeneous reagent; and (ii) a reaction time that is less than a threshold reaction time for the Lewis-acidic heterogeneous reagent and the reaction temperature.

In select embodiments, the present disclosure relates to a method for converting CBD into primarily Δ⁹-THC. In such embodiments, the method may comprise contacting the CBD with a Lewis-acidic heterogeneous reagent under neat reaction conditions comprising: (i) a reaction temperature that is less than a threshold reaction temperature for the Lewis-acidic heterogeneous reagent; and (ii) a reaction time that is less than a threshold reaction time for the Lewis-acidic heterogeneous reagent and the reaction temperature.

In select embodiments, the present disclosure relates to a method for converting CBD into a composition comprising Δ⁹-THC and Δ⁸-THC, in which the composition has a Δ⁹-THC:Δ⁸-THC ratio that is greater than 1.0:1.0. In such embodiments, the method may comprise contacting the CBD with an aluminosilicate-based Lewis-acidic heterogeneous reagent under reaction conditions comprising: (i) an aprotic-solvent system comprising heptane; (ii) a reaction temperature that is less than 65° C.; and (iii) a reaction time that is less 24 hours.

In select embodiments, the present disclosure relates to a method for converting CBD into a composition comprising Δ⁹-THC and Δ⁸-THC, in which the composition has a Δ⁹-THC:Δ⁸-THC ratio that is greater than 1.0:1.0. In such embodiments, the method may comprise contacting the CBD with an aluminosilicate-based Lewis-acidic heterogeneous reagent under neat reaction conditions comprising: (i) a reaction temperature that is less than 80° C.; and (ii) a reaction time that is less 1 hour.

Other aspects and features of the present disclosure will become apparent to those ordinarily skilled in the art upon review of the following description of specific embodiments.

### BRIEF DESCRIPTION OF THE DRAWINGS

FIG. 1 shows a high-performance liquid chromatogram for EXAMPLE 1.

FIG. 2 shows a high-performance liquid chromatogram for COMPARISON EXAMPLE 1.

FIG. 3 shows a high-performance liquid chromatogram for COMPARISON EXAMPLE 2.

FIG. 4 shows a high-performance liquid chromatogram for EXAMPLE 2.

FIG. 5 shows a high-performance liquid chromatogram for COMPARISON EXAMPLE 3.

FIG. 6A shows the effect of ZSM-5 silica/alumina ratio on CBD conversion.

FIG. 6B shows the effect of ZSM-5 silica/alumina ratio on Δ⁹-THC:Δ⁸-THC ratio.

FIG. 7A shows the effect of water and isopropyl alcohol (IPA) on CBD conversion to Δ⁹-tetrahydrocannabinol and/or mixtures of Δ⁹-tetrahydrocannabinol and Δ⁸-tetrahydrocannabinol.

FIG. 7B shows the effect of butylated hydroxyanisole (BHA) on CBD conversion.

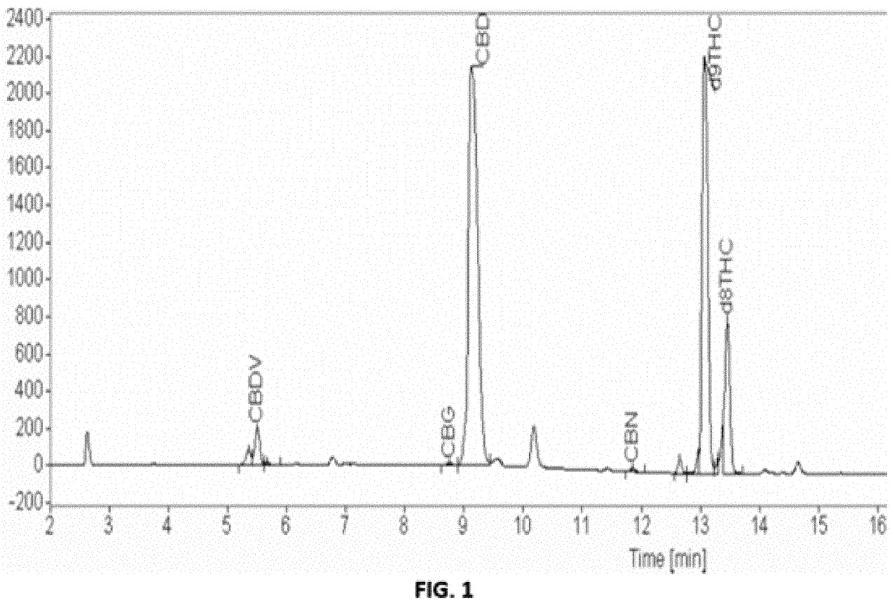

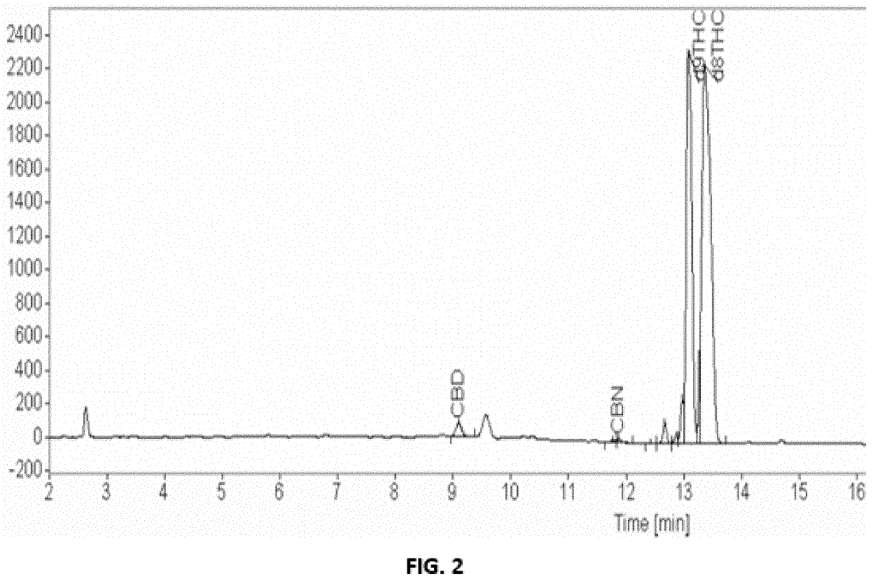

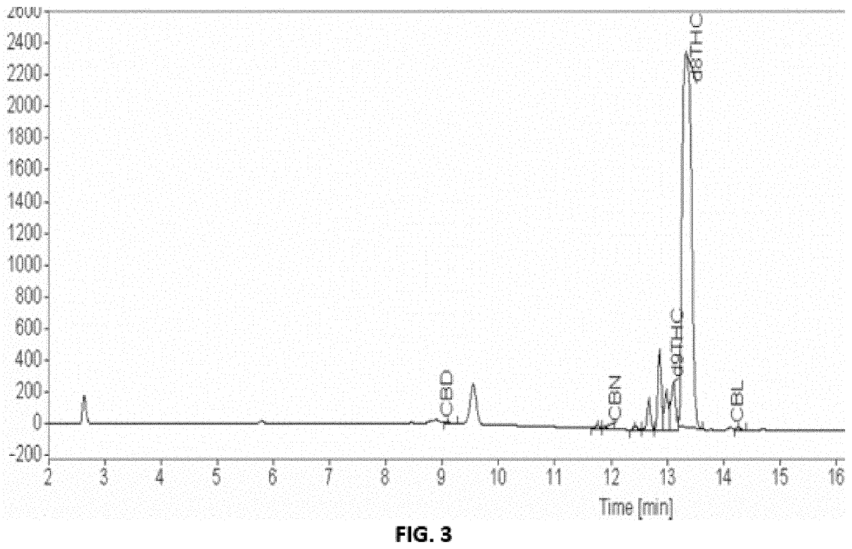

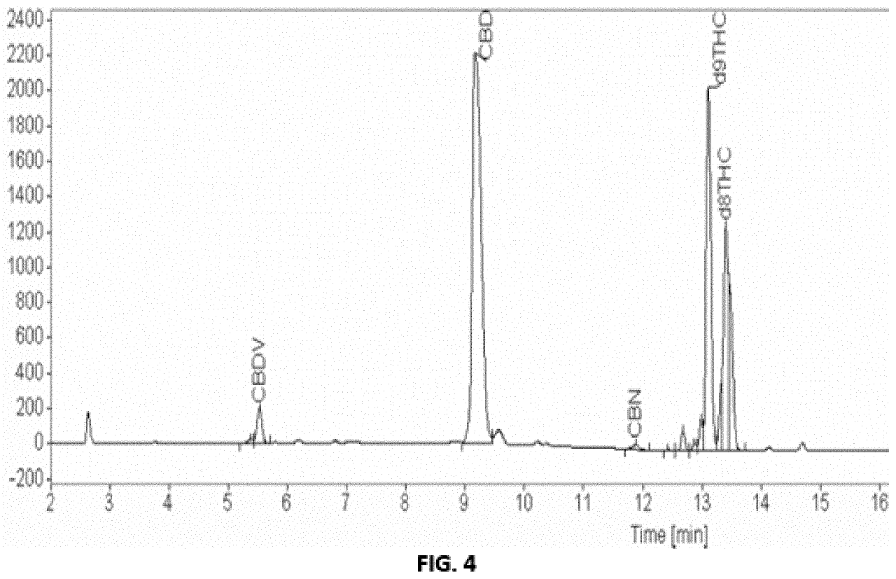

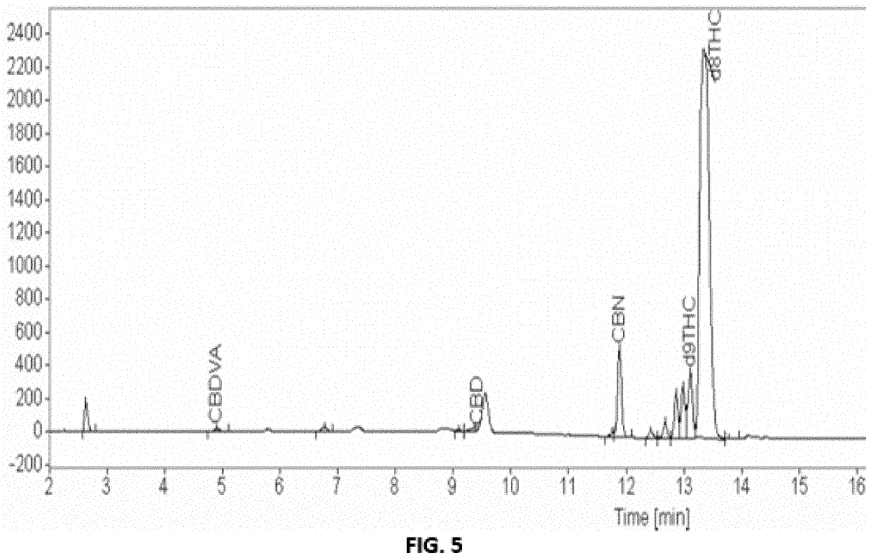

### DETAILED DESCRIPTION

As noted above, the present disclosure provides improved methods of converting cannabidiol (CBD) into primarily Δ⁹-tetrahydrocannabinol (Δ⁹-THC) and/or mixtures of Δ⁹-THC and Δ⁸-tetrahydrocannabinol (Δ⁸-THC) having Δ⁹-THC:Δ⁸-THC ratios of greater than 1.0:1.0. The methods of the present disclosure are suitable for use at industrial scale in that they do not require: (i) complicated and/or dangerous reagent-addition, quenching, and/or work-up steps; and (ii) dangerous and/or toxic solvents and/or reagents. Importantly, the methods of the present disclosure provide access to compositions with numerous Δ⁹-THC:Δ⁸-THC ratios above 1.0:1.0 as evidenced by examples disclosed herein. For example, a first Lewis-acidic heterogeneous reagent and a first set of reaction conditions disclosed herein provide a Δ⁹-THC:Δ⁸-THC ratio of about 3.1:1.0, while a second Lewis-acidic reagent and a second set of reaction conditions disclosed herein provide a Δ⁹-THC:Δ⁸-THC ratio of about 1.7:1.0. Because the Δ⁹-THC:Δ⁸-THC ratios disclosed herein can be correlated to particular reaction conditions and reagents, the methods of the present disclosure may be tuned towards particular Δ⁹-THC/Δ⁸-THC selectivity outcomes. While there may be little information available in the current research literature on the pharmacokinetic interactions between Δ⁹-THC and Δ⁸-THC, the present disclosure asserts that access to such compositions is desirable in both medicinal and recreational contexts. Moreover, the present disclosure asserts that access to an array of compositions of varying Δ⁹-THC:Δ⁸-THC ratios may also desirable to synthetic chemists.

Without being bound to any particular theory, the present disclosure reports that the ability to form Δ⁹-THC and/or compositions of various Δ⁹-THC:Δ⁸-THC ratios greater than 1.0:1.0 (as demonstrated herein) is associated with the utilization of Lewis-acidic heterogeneous reagents in aprotic-solvent systems, or under neat reaction conditions, in which reaction temperature and reaction time parameters are carefully selected and controlled. In particular, the examples of the present disclosure indicate that for either neat reaction conditions or reactions in aprotic-solvent systems, mild reaction temperatures and short reaction times favor the formation of Δ⁹-THC over Δ⁸-THC and that the properties of the Lewis-acidic heterogeneous reagent affect the selection of such reaction conditions. The examples disclosed herein also indicate that the application of Lewis-acidic heterogeneous reagents to the conversion of CBD to primarily Δ⁹-THC is compatible with the use of neat reaction conditions or aprotic-solvent systems provided the reaction conditions are carefully selected and controlled. The use of neat reaction conditions or aprotic-solvent systems for such transformations may obviate the need for the dangerous and/or hazardous solvents that are typical of the prior art. The utilization of Lewis-acidic heterogeneous reagents may also allow product mixtures to be isolated by simple solid/liquid separations (e.g. filtration and/or decantation). As such, the combination of Lewis-acidic heterogeneous reagents and neat reaction conditions or aprotic-solvent systems appears to underlie one more of the advantages of the present disclosure.

In select embodiments, the present disclosure relates to a method for converting CBD into a composition comprising Δ⁹-THC and Δ⁸-THC, wherein the composition has a Δ⁹-THC:Δ⁸-THC ratio that is greater than 1.0:1.0, the method comprising contacting the CBD with a Lewis-acidic heterogeneous reagent under reaction conditions comprising: (i) an aprotic-solvent system; (ii) a reaction temperature that is less than a threshold reaction temperature for the Lewis-acidic heterogeneous reagent and the aprotic-solvent system; and (iii) a reaction time that is less than a threshold reaction time for the Lewis-acidic heterogeneous reagent, the aprotic-solvent system, and the reaction temperature.

In select embodiments, the present disclosure relates to a method for converting CBD into primarily Δ⁹-THC, the method comprising contacting the CBD with a Lewis-acidic heterogeneous reagent under reaction conditions comprising: (i) an aprotic-solvent system; (ii) a reaction temperature that is less than a threshold reaction temperature for the Lewis-acidic heterogeneous reagent and the aprotic-solvent system; and (iii) a reaction time that is less than a threshold reaction time for the Lewis-acidic heterogeneous reagent, the aprotic-solvent system, and the reaction temperature.

In select embodiments, the present disclosure relates to a method for converting CBD into a composition comprising Δ⁹-THC and Δ⁸-THC, wherein the composition has a Δ⁹-THC:Δ⁸-THC ratio that is greater than 1.0:1.0, the method comprising contacting the CBD with a Lewis-acidic heterogeneous reagent under neat reaction conditions comprising: (i) a reaction temperature that is less than a threshold reaction temperature for the Lewis-acidic heterogeneous reagent; and (ii) a reaction time that is less than a threshold reaction time for the Lewis-acidic heterogeneous reagent and the reaction temperature.

In select embodiments, the present disclosure relates to a method for converting CBD into primarily Δ⁹-THC, the method comprising contacting the CBD with a Lewis-acidic heterogeneous reagent under neat reaction conditions comprising: (i) a reaction temperature that is less than a threshold reaction temperature for the Lewis-acidic heterogeneous reagent; and (ii) a reaction time that is less than a threshold reaction time for the Lewis-acidic heterogeneous reagent and the reaction temperature.

In select embodiments, the present disclosure relates to a method for converting CBD into a composition comprising Δ⁹-THC and Δ⁸-THC, in which the composition has a Δ⁹-THC:Δ⁸-THC ratio that is greater than 1.0:1.0, the method comprising contacting the CBD with an aluminosilicate-based Lewis-acidic heterogeneous reagent under reaction conditions comprising: (i) an aprotic-solvent system comprising heptane; (ii) a reaction temperature that is less than 65° C.; and (iii) a reaction time that is less 24 hours.

In select embodiments, the present disclosure relates to a method for converting CBD into a composition comprising Δ⁹-THC and Δ⁸-THC, wherein the composition has a Δ⁹-THC:Δ⁸-THC ratio that is greater than 1.0:1.0, the method comprising contacting the CBD with an aluminosilicate-based Lewis-acidic heterogeneous reagent under neat reaction conditions comprising: (i) a reaction temperature that is less than 80° C.; and (ii) a reaction time that is less than 1 hour.

In the context of the present disclosure, the term “contacting” and its derivatives is intended to refer to bringing the CBD and the Lewis-acidic heterogeneous reagent as disclosed herein into proximity such that a chemical reaction can occur. In some embodiments of the present disclosure, the contacting may be by adding the heterogeneous catalyst to the CBD. In some embodiments, the contacting may be by combining, mixing, or both.

In the context of the present disclosure, the term “CBD” refers to cannabidiol or, more generally, cannabidiol-type cannabinoids. Accordingly the term “CBD” includes: (i) acid forms, such as “A-type”, “B-type”, or “AB-type” acid forms; (ii) salts of such acid forms, such as Na⁺ or Ca²⁺ salts of such acid forms; (iii) ester forms, such as formed by hydroxyl-group esterification to form traditional esters, sulphonate esters, and/or phosphate esters; (iv) various double-bond isomers, such as Δ¹-CBD and Δ⁶-CBD as well as cis/trans isomers thereof; and/or (v) various stereoisomers. In select embodiments of the present disclosure, the CBD is a component of a distillate, an isolate, a concentrate, an extract, or a combination thereof. In select embodiments of the present disclosure, CBD may have the following structural formula:

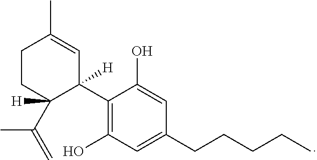 [SMILES: C=C(C)[C@@H]1CCC(C)=C[C@H]1c1c(O)cc(CCCCC)cc1O]

In the context of the present disclosure, the term “Δ⁹-THC” refers to Δ⁹-tetrahydrocannabinol or, more generally, Δ⁹-tetrahydrocannabinol-type cannabinoids. Accordingly the term “Δ⁹-THC” includes: (i) acid forms, such as “A-type”, “B-type”, or “AB-type” acid forms; (ii) salts of such acid forms, such as Na⁺ or Ca²⁺ salts of such acid forms; (iii) ester forms, such as those formed by hydroxyl-group esterification to form traditional esters, sulphonate esters, and/or phosphate esters; and/or (iv) various stereoisomers. Δ⁹-THC may have the following structural formula:

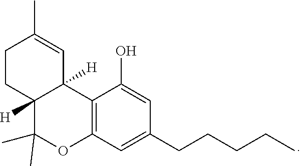 [SMILES: CCCCCc1cc(O)c2c(c1)OC(C)(C)[C@@H]1CCC(C)=C[C@@H]21]

In the context of the present disclosure, the term “Δ⁸-THC” refers to Δ⁸-tetrahydrocannabinol or, more generally, Δ⁸-tetrahydrocannabinol-type cannabinoids. Accordingly the term “Δ⁸-THC” includes: (i) acid forms, such as “A-type”, “B-type”, or “AB-type” acid forms; (ii) salts of such acid forms, such as Na⁺ or Ca²⁺ salts of such acid forms; and/or (iii) ester forms, such as those formed by hydroxyl-group esterification to form traditional esters, sulphonate esters, and/or phosphate esters; and/or (iv) various stereoisomers. In select embodiments of the present disclosure, Δ⁸-THC may have the following structural formula:

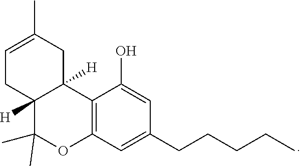 [SMILES: CCCCCc1cc(O)c2c(c1)OC(C)(C)[C@@H]1CC=C(C)C[C@@H]21]

In the context of the present disclosure, the relative quantities of Δ⁹-THC and Δ⁸-THC in a particular composition may be expressed as a ratio—Δ⁹-THC:Δ⁸-THC. Those skilled in the art will recognize that a variety of analytical methods may be used to determine such ratios, and the protocols required to implement any such method are within the purview of those skilled in the art. By way of non-limiting example, Δ⁹-THC:Δ⁸-THC ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or combinations thereof. In select embodiments of the present disclosure, the compositions provided by the methods of the present disclosure have Δ⁹-THC:Δ⁸-THC ratios of greater than 1.0:1.0, meaning the quantity of Δ⁹-THC in the composition is greater than the quantity of Δ⁸-THC in the composition. For example, the compositions provided by the methods of the present disclosure may have Δ⁹-THC:Δ⁸-THC ratios of: (i) greater than about 2.0:1.0; (ii) greater than about 3.0:1.0; (iii) greater than about 5.0:1.0; (iv) greater than about 10.0:1.0; (v) greater than about 15.0:1.0; (vi) greater than about 20.0:1.0; (vii) greater than about 50.0:1.0; or (viii) greater than about 100.0:1.0.

In the context of the present disclosure, converting CBD into “primarily” Δ⁹-THC refers to converting CBD into exclusively Δ⁹-THC or into a composition in which Δ⁹-THC is present to a greater extent than any other reaction product. In select embodiments of the present disclosure, converting CBD into “primarily” Δ⁹-THC may yield a product mixture which is at least: (i) 50% Δ⁹-THC on a molar basis; (ii) 60% Δ⁹-THC on a molar basis; (iii) 70% Δ⁹-THC on a molar basis; (iv) 80% Δ⁹-THC on a molar basis; (v) 90% Δ⁹-THC on a molar basis; or (vi) 95% Δ⁹-THC on a molar basis. Importantly converting CBD into a composition in which Δ⁹-THC is the primary product does not necessarily imply that CBD is the most prevalent component of a reaction composition, as other constituents derived from the starting material may be more prevalent. For example, Δ⁹-THC may be the primary product in a reaction mixture that includes primarily unreacted CBD.

In the context of the present disclosure, a Lewis-acid heterogeneous reagent is one which: (i) comprises one or more sites that are capable of accepting an electron pair from an electron pair donor; and (ii) is substantially not mono-phasic with the reagent (i.e. CBD). Likewise, in the context of the present disclosure, a Brønsted-acid heterogeneous reagent is one which: (i) comprises one or more sites that are capable of donating a proton to a proton-acceptor; and (ii) is substantially not mono-phasic with the starting material and/or provides an interface where one or more chemical reaction takes place. Importantly, the term “reagent” is used in the present disclosure to encompass both reactant-type reactivity (i.e. wherein the reagent is at least partly consumed as reactant is converted to product) and catalyst-type reactivity (i.e. wherein the reagent is not substantially consumed as reactant is converted to product).

In the context of the present disclosure, the acidity of a Lewis-acid heterogeneous reagent and/or a Brønsted-acid heterogeneous reagent may be characterized by a variety of parameters, non-limiting examples of which are summarized in the following paragraphs.

As will be appreciated by those skilled in the art who have benefitted from the teachings of the present disclosure, determining the acidity of heterogeneous solid acids may be significantly more challenging than measuring the acidity of homogenous acids due to the complex molecular structure of heterogeneous solid acids. The Hammett acidity function (H₀) has been applied over the last 60 years to characterize the acidity of solid acids in non-aqueous solutions. This method utilizes organic indicator bases, known as Hammett indicators, which coordinate to the accessible acidic sites of the solid acid upon protonation. Typically, a color change is observed during titration with an additional organic base (e.g. n-butylamine), which is measured by UV-visible spectroscopy to quantify acidity. Multiple Hammett indicators with pKa values ranging from +6.8 (e.g. neutral red) to −8.2 (e.g. anthraquinone) are tested with a given solid acid to determine the quantity and strength of acidic sites, which is typically expressed in mmol per gram of solid acid for each indicator. Hammett acidity values may not provide a complete characterization of acidity. For example, accurate measurement of acidity may rely on the ability of the Hammett indicator to access the interior acidic sites within the solid acid. Some solid acids may have pore sizes that permit the passage of small molecules but prevent larger molecules from accessing the interior of the acid. H-ZSM-5 may be a representative example, wherein larger Hammett indicators such as anthraquinone may not be able to access interior acidic sites, which may lead to an incomplete measure of its total acidity.

Temperature-Programmed Desorption (TPD) is an alternate technique for characterizing the acidity of heterogeneous solid acids. This technique typically utilizes an organic base with small molecular size (e.g. ammonia, pyridine, n-propylamine), which may react with the acid sites on the exterior and interior of the solid acid in a closed system. After the solid acid is substantially saturated with organic base, the temperature is increased and the change in organic base concentration is monitored gravimetrically, volumetrically, by gas chromatography, or by mass spectrometry. The amount of organic base desorbing from the solid acid above some characteristic temperature may be interpreted as the acid-site concentration. TPD is often considered more representative of total acidity for solid acids compared to the Hammett acidity function, because the selected organic base is small enough to bind to acidic sites on the interior of the solid acid.

In select embodiments of the present disclosure, TPD values are reported with respect to ammonia. Those skilled in the art who have benefited from the teachings of the present disclosure will appreciate that ammonia may have the potential disadvantage of overestimating acidity, because its small molecular size enables access to acidic sites on the interior of the solid acid that are not accessible to typical organic substrates being employed for chemical reactions (i.e. ammonia may fit into pores that CBD cannot). Despite this disadvantage, TPD with ammonia is still considered a useful technique to compare total acidity of heterogeneous solid acids (larger NH₃ absorption values correlate with stronger acidity).

Another commonly used method for characterizing the acidity of heterogeneous solid acids is microcalorimetry. In this technique, the heat of adsorption is measured when acidic sites on the solid acid are neutralized by addition of a base. The measured heat of adsorption is used to characterize the strength of Brønsted-acid sites (the larger the heat of adsorption, the stronger the acidic site, such that more negative values correlate with stronger acidity).

Microcalorimetry may provide the advantage of being a more direct method for the determination of acid strength when compared to TPD. However, the nature of the acidic sites cannot be determined by calorimetry alone, because adsorption may occur at Brønsted sites, Lewis sites, or a combination thereof. Further, experimentally determined heats of adsorption may be inconsistent in the literature for a given heterogeneous acid. For example, ΔH_{0ads} NH₃ values between about 100 kJ/mol and about 200 kJ/mol have been reported for H-ZSM-5. Thus, heats of adsorption determined by microcalorimetry may be best interpreted in combination with other acidity characterization methods such as TPD to properly characterize the acidity of solid heterogeneous acids.

Non-limiting examples of: (i) Hammett acidity values; (ii) TPD values with reference to ammonia; and (iii) microcalorimetry values with reference to ammonia, for a selection of Lewis-acidic heterogeneous reagents in accordance with the present disclosure are set out in Table 1.

TABLE 1
Non-limiting examples of: (i) Hammett acidity values;
(ii) TPD values with reference to ammonia; and (iii)
microcalorimetry values with reference to ammonia.

| | | | | ΔH⁰_{ads} |
|---|---|---|---|---|
| | | Hammett Value | TPD NH₃ | NH₃ |
| Acid Reagent | Classification | (H₀) | (mmol/g) | (kJ/mol) |
| Amberlyst-35 | Ion-exchange | −5.6 | 5.2^{]} | −117 |
| | resin | | | |
| Amberlyst-15 | Ion-exchange | −4.6 | 4.6 | −116 |
| | resin | | | |
| H-ZSM-5 | Microporous | −5.6 < H₀ < | 1.0^{]} | −145 |
| | aluminosilicate | −3.0 | | |
| | (zeolite) | | | |
| H-Beta | Microporous | — | 0.65 | −120 |
| | aluminosilicate | | | |
| | (zeolite) | | | |
| Al-MCM-41 | Mesoporous | —. | 0.26 | —. |
| | aluminosilicate | | | |
| Montmorillonite | Phyllosilicate | −1.5 < H₀ < | 0.18 | — |
| (K30) | (clay) | +3.2 | | |

In select embodiments of the present disclosure, the Lewis-acidic heterogeneous reagent may have a Hammett-acidity value (Hₒ) of between about −8.0 and about 0.0. For example, the Lewis-acidic heterogeneous reagent may have a Hammett-acidity value (Hₒ) of between: (i) about −8.0 and about −7.0; (ii) about −7.0 and about −6.0; (iii) about −6.0 and about −5.0; (iv) about −5.0 and about −4.0; (v) about −4.0 and about −3.0; (vi) about −3.0 and about −2.0; (vii) about −2.0 and about −1.0; or (viii) about −1.0 and about 0.

In select embodiments of the present disclosure, the Lewis-acidic heterogeneous reagent may have a temperature-programmed desorption value of between about 7.5 and about 0.0 as determined with reference to ammonia (TPD_{NH3}). For example, the Lewis-acidic heterogeneous reagent may have a temperature-programmed desorption value of between: (i) about 7.5 and about 6.5 as determined with reference to ammonia (TPD_{NH3}); (ii) about 6.5 and about 5.5 as determined with reference to ammonia (TPD_{NH3}); (iii) about 5.5 and about 4.5 as determined with reference to ammonia (TPD_{NH3}); (iv) about 4.5 and about 3.5 as determined with reference to ammonia (TPD_{NH3}); (v) about 3.5 and about 2.5 as determined with reference to ammonia (TPD_{NH3}); (vi) about 2.5 and about 1.5 as determined with reference to ammonia (TPD_{NH3}); (vii) about 1.5 and about 0.5 as determined with reference to ammonia (TPD_{NH3}); or (viii) about 0.5 and about 0.0 as determined with reference to ammonia (TPD_{NH3}).

In select embodiments of the present disclosure, the Lewis-acidic heterogeneous reagent may have a heat of absorption value of between about −165 and about −100 as determined with reference to ammonia (ΔH^{o}_{ads NH3}). For example, the Lewis-acidic heterogeneous reagent may have a heat of absorption value of between: (i) about −165 and about −150 as determined with reference to ammonia (ΔH^{o}_{ads NH3}); (ii) about −150 and about −135 as determined with reference to ammonia (ΔH^{o}_{ads NH3}); (iii) about −135 and about −120 as determined with reference to ammonia (ΔH^{o}_{ads NH3}); (iv) about −120 and about −105 as determined with reference to ammonia (ΔH^{o}_{ads NH3}); or (v) about −105 and about −100 as determined with reference to ammonia (ΔH^{o}_{ads NH3}).

In select embodiments of the present disclosure, the Lewis-acidic heterogeneous reagent may comprise an ion-exchange resin, a microporous silicate, a mesoporous silicate, and/or a phyllosilicate.

Lewis-acidic heterogeneous reagents that comprise an ion-exchange resin may comprise an Amberlyst polymeric resins. Amberlyst polymeric resins include but are not limited to Amberlyst-15, 16, 31, 33, 35, 36, 39, 46, 70, CH10, CH28, CH43, M-31, wet forms, dry forms, macroreticular forms, gel forms, H⁺ forms, Na⁺ forms, or combinations thereof). In select embodiments of the present disclosure, the Lewis-acidic heterogeneous reagent may comprise an Amberlyst resin that has a surface area of between about 20 m²/g and about 80 m²/g. In select embodiments of the present disclosure, the Lewis-acidic heterogeneous reagent may comprise an Amberlyst resin that has an average pore diameter of between about 100 Å and about 500 Å. In select embodiments of the present disclosure, the Lewis-acidic heterogeneous reagent may comprise Amberlyst-15. Amberlyst-15 is a styrene-divinylbenzene-based polymer with sulfonic acid functional groups linked to the polymer backbone. Amberlyst-15 may have the following structural formula:

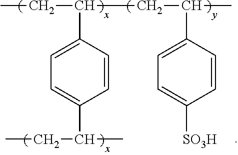

Lewis-acidic heterogeneous reagents that comprise an ion-exchange resin may comprise a Nafion polymeric resin. Nafion polymeric resins may include but are not limited to Nafion-NR50, N115, N117, N324, N424, N1110, SAC-13, powder forms, resin forms, membrane forms, aqueous forms, dispersion forms, composite forms, H⁺ forms, Na⁺ forms, or combinations thereof.

Lewis-acidic heterogeneous reagents that comprise microporous silicates (e.g. zeolites) may comprise, for example, natural and synthetic zeolites. Lewis-acidic heterogeneous reagents that comprise mesoporous silicates may comprise, for example, Al-MCM-41 and/or MCM-41. Lewis-acidic heterogeneous reagents that comprise phyllosilicates may comprise, for example, montmorillonite. A commonality amongst these materials is that they are all silicates. Silicates may include but are not limited to Al-MCM-41, MCM-41, MCM-48, SBA-15, SBA-16, ZSM-5, ZSM-11, ZSM-22, ZSM-23, ZSM-35, SAPO-11, SAPO-34, SSZ-13, TS-1, KIT-5, KIT-6, FDU-12, Beta, X-type, Y-type, Linde type A, Linde type L, Linde type X, Linde type Y, Faujasite, USY, Mordenite, Ferrierite, Montmorillonite K10, K30, KSF, Clayzic, bentonite, H⁺ forms, Na⁺ forms, or combinations thereof. Zeolites are commonly used as adsorbents and catalysts (e.g. in fluid catalytic cracking and hydrocracking in the petrochemical industry). Although zeolites are abundant in nature, the zeolites used for commercial and industrial processes are often made synthetically. Their structural framework consists of SiO₄ and AlO₄⁻ tetrahedra, which are combined in specific ratios with an amine or tetraalkylammonium salt “template” to give a zeolite with unique acidity, shape and pore size. The Lewis and/or Brønsted-Lowry acidity of zeolites can typically be modified using two approaches. One approach involves adjusting the Si/Al ratio. Since an AlO₄⁻ moiety is unstable when attached to another AlO₄⁻ unit, it is necessary for them to be separated by at least one SiO₄ unit. The strength of the individual acidic sites may increase as the AlO₄⁻ units are further separated Another approach involves cation exchange. Since zeolites contain charged AlO₄⁻ species, an extra-framework cation such as Na⁺ is required to maintain electroneutrality. The extra-framework cations can be replaced with protons to generate the “H-form” zeolite, which has stronger Brønsted acidity than its metal cation counterpart.

In select embodiments of the present disclosure, the Lewis-acidic heterogeneous reagent may comprise “H⁺-form” zeolites “Na⁺-form” zeolites, and/or a suitable mesoporous material. By way of non-limiting example, the acidic heterogeneous reagent may comprise Al-MCM-41, MCM-41, MCM-48, SBA-15, SBA-16, ZSM-5, ZSM-11, ZSM-22, ZSM-23, ZSM-35, SAPO-11, SAPO-34, SSZ-13, TS-1, KIT-5, KIT-6, FDU-12, Beta, X-type, Y-type, Linde type A, Linde type L, Linde type X, Linde type Y, Faujasite, USY, Mordenite, Ferrierite, Montmorillonite, Bentonite, or combinations thereof. Suitable mesoporous materials and zeolites may have a pore diameter ranging from about 0.1 nm to about 100 nm, particle sizes ranging from about 0.1 μm to about 50 μm, Si/Al ratio ranging from 5-1500, and any of the following cations: H⁺, Na⁺, K⁺, NH₄⁺, Rb⁺, Cs⁺, Ag⁺. Furthermore, suitable zeolites may have frameworks that are substituted with or coordinated to other atoms including, for example, titanium, copper, iron, cobalt, manganese, chromium, zinc, tin, zirconium, and gallium.

In select embodiments of the present disclosure, the Lewis-acidic heterogeneous reagent is H-ZSM-5 (P-38 (Si/Al=38), H⁺ form, ˜5 angstrom pore size, 2 μm particle size), Na-ZSM-5 (P-38 (Si/Al=38), Na⁺ form, ˜5 angstrom pore size, 2 μm particle size), Al-MCM-41 (aluminum-doped Mobil Composition of Matter No. 41; e.g., P-25 (Si/Al=25), 2.7 nm pore diameter), or combinations thereof.

In select embodiments of the present disclosure, the ZSM-5 has a silica to alumina ratio (molecular ratio, MR) that may be selected to control the Δ⁹-THC:Δ⁸-THC ratio, the percent CBD conversion, or both.

In select embodiments of the present disclosure, the Lewis-acidic heterogeneous reagent may be acidic alumina. Acidic alumina is also known as activated alumina and is a highly porous aluminum oxide often used in chromatography separation of, for example, phenols, sulphonic acids, carboxylic acids and amino acids.

In embodiments where the Lewis-acidic heterogeneous reagent is acidic alumina, the CBD may be comprised in a CBD isolate. In these embodiments, the method may further comprise adding an additive in an amount of about 1% w/w to about 5% w/w. In some embodiments, the additive is added in an amount of about 3% w/w. In select embodiments, the additive is water, isopropanol, or a combination thereof. Without being bound by a particular theory, the addition of an additive may influence the Δ⁹-THC:Δ⁸-THC ratio.

In select embodiments of the present disclosure, CBD is contacted with a Lewis-acidic heterogeneous reagent in an aprotic-solvent system. In the context of the present disclosure, aprotic solvent systems may comprise dimethyl sulfoxide, ethyl acetate, dichloromethane, chloroform, toluene, pentane, heptane, hexane, diethyl ether, tert-butyl methyl ether, tetrahydrofuran, dioxane, dimethylformamide, dimethylacetamide, N-methylpyrrolidone, anisole, butyl acetate, cumene, ethyl formate, isobutyl acetate, isopropyl acetate, methyl acetate, methylethylketone, methylisobutylketone, propyl acetate, cyclohexane, para-xylene, meta-xylene, ortho-xylene, 1,2-dichloroethane, or a combination thereof. In select embodiments of the present disclosure, the aprotic-solvent system may comprise a class III solvent. Heptane is a non-limiting example of an aprotic class III solvent.

In select embodiments of the present disclosure, CBD is contacted with a Lewis-acidic heterogeneous reagent under neat reaction conditions. As will be appreciated by those skilled in the art who have benefitted from the teachings of the present disclosure, neat reaction conditions are those which do not include exogenous solvent, but may include solvent-like components that are derived from the reactant composition.

In select embodiments of the present disclosure, CBD is contacted with a Lewis-acidic reagent under reaction conditions characterized by: (i) a reaction temperature that is less than a threshold reaction temperature for the particular Lewis-acidic heterogeneous reagent and optionally the particular aprotic-solvent system; and (ii) a reaction time that is less than a threshold reaction time for the particular Lewis-acidic heterogeneous reagent, optionally the particular aprotic-solvent system, and the particular reaction temperature. As evidenced by the examples of the present disclosure, the acidity of the Lewis-acidic heterogeneous reagent and the characteristics of the aprotic-solvent system impact the threshold reaction-temperature and the threshold reaction time. Without being bound to any particular theory, the examples of the present disclosure appear to indicate that particular Lewis-acidic heterogeneous reagents, milder reaction temperatures, and/or shorter reaction times appear to favor Δ⁹-THC formation over Δ⁸-THC formation. Importantly, these reaction parameters appear to be dependent variables in that altering one may impact the others. As such, each reaction temperature may be considered in reference to a threshold reaction temperature for the particular Lewis-acidic heterogeneous reagent, the particular aprotic-solvent system, and the particular reaction time associated with the reaction. Likewise, each reaction time in the present disclosure may be considered in reference to a threshold reaction time for the particular Lewis-acidic heterogeneous reagent, the particular aprotic-solvent system, and the particular reaction temperature. With respect to reaction temperatures, by way of non-limiting example, methods of the present disclosure may involve reaction temperatures ranging from about 0° C. to about 200° C. For example, methods of the present disclosure may involve reaction temperatures between: (i) about 5° C. and about 15° C.; (ii) about 15° C. and about 25° C.; (iii) about 25° C. and about 35° C.; (iv) about 35° C. and about 45° C.; (v) about 45° C. and about 55° C.; (vi) about 55° C. and about 65° C.; (vii) about 65° C. and about 75° C.; (viii) about 75° C. and about 85° C.; (ix) about 85° C. and about 95° C.; (x) about 95° C. and about 105° C.; (xi) about 105° C. and about 115° C.; or a combination thereof. Of course, the reaction temperature may be varied over the course of the reaction while still being characterized the one or more of the foregoing reaction temperatures. With respect to reaction times, by way of non-limiting example, methods of the present disclosure may involve reaction temperatures ranging from about 10 minutes to about 85 hours. For example, methods of the present disclosure may involve reaction times between: (i) 10 minutes and about 1 hour; (ii) about 1 hour and about 5 hours; (iii) about 5 hours and about 10 hours; (iv) about 10 hours and 25 hours; (v) about 25 hours and about 40 hours; (vi) about 40 hours and about 55 hours; (vii) about 55 hours and about 70 hours; or (viii) about 70 hours and about 85 hours.

In select embodiments, methods of the present disclosure may involve reactant (i.e. CBD) concentrations ranging from about 0.001 M to about 2 M. For example methods of the present disclosure may involve reactant concentrations of: (i) between about 0.01 M and about 0.1 M; (ii) between about 0.1 M and about 0.5 M; (iii) between about 0.5 M and about 1.0 M; (iv) between about 1.0 M and about 1.5 M; or (v) between about 1.5 M and about 2.0 M.

In select embodiments, methods of the present disclosure may involve Lewis-acidic heterogeneous reagent loadings ranges from about 0.1 molar equivalents to about 100 molar equivalents relative to the reactant (i.e. CBD). For example methods of the present disclosure may involve Lewis-acidic heterogeneous reagent loadings of: (i) between about 0.1 molar equivalents to about 1.0 molar equivalents, relative to the reactant; (ii) .1.0 molar equivalents to about 5.0 molar equivalents, relative to the reactant; (iii) 5.0 molar equivalents to about 10.0 molar equivalents, relative to the reactant; (iv) 10.0 molar equivalents to about 50.0 molar equivalents, relative to the reactant; or (v) 50.0 molar equivalents to about 100.0 molar equivalents, relative to the reactant.

In select embodiments, the methods of the present disclosure may produce an amount of exo-tetrahydrocannabinol (exo-THC). In select embodiments, the amount of exo-THC is detectable by HPLC. In select embodiments, the formation of exo-THC may be directly related to the Brønsted-acidity of the Lewis-acid heterogeneous reagent. In the context of the present disclosure, exo-THC may have the following structure:

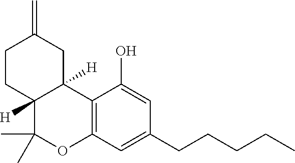 [SMILES: C=C1CC[C@@H]2[C@@H](C1)c1c(O)cc(CCCCC)cc1OC2(C)C]

In select embodiments, the methods of the present disclosure may further comprise a filtering step. By way of non-limiting example the filtering step may employ a fritted Buchner filtering funnel. Suitable filtering apparatus and protocols are within the purview of those skilled in the art.

In select embodiments, the methods of the present disclosure may further comprise a solvent evaporation step, and the solvent evaporation step may be executed under reduced pressure (i.e. in vacuo) for example with a rotary evaporator. Suitable evaporating apparatus and protocols are within the purview of those skilled in the art.

In select embodiments, the methods of the present disclosure may further comprise a step of distillation. Without being bound by any particular theory, distillation may remove impurities and result in a composition comprising a total cannabinoid content about equal to the total cannabinoid content prior to undergoing one of the methods disclosed herein. Suitable distillation apparatus and protocols are within the purview of those skilled in the art.

### Exemplary Embodiments

(1) A method for converting cannabidiol (CBD) into a composition comprising Δ9-tetrahydrocannabinol (Δ9-THC) and Δ8-tetrahydrocannabinol (Δ8-THC), wherein the composition has a Δ9-THC:Δ8-THC ratio that is greater than 1.0:1.0, the method comprising contacting the CBD with a Lewis-acidic heterogeneous reagent under reaction conditions comprising: (i) an aprotic-solvent system; (ii) a reaction temperature that is less than a threshold reaction temperature for the Lewis-acidic heterogeneous reagent and the aprotic-solvent system; and (iii) a reaction time that is less than a threshold reaction time for the Lewis-acidic heterogeneous reagent, the aprotic-solvent system, and the reaction temperature.

(2) The method of (1), wherein the Lewis-acidic heterogeneous reagent is a Brønsted-acidic heterogeneous reagent.

(3) The method of (1) or (2), wherein the Lewis-acidic heterogeneous reagent has a Hammett-acidity value (Ho) of between about −8.0 and about 0.0.

(4) The method of any one of (1) to (3), wherein the Lewis-acidic heterogeneous reagent has a temperature-programmed desorption value of between about 7.5 and about 0.0 as determined with reference to ammonia (TPDNH3).

(5) The method of any one of (1) to (4), wherein the Lewis-acidic heterogeneous reagent has a heat of absorption value of between about −165 and about −100 as determined with reference to ammonia (ΔHoads NH3).

(6) The method of (1), wherein the Lewis-acidic heterogeneous reagent comprises an ion-exchange resin, a microporous silicate, a mesoporous silicate, a phyllosilicate, or a combination thereof.

(7) The method of (6), wherein the ion-exchange resin is an Amberlyst polymeric resin.

(8) The method of (7), wherein the Amberlyst polymeric resin has a surface area of between about 20 m2/g and about 80 m2/g and an average pore diameter of between about 100 Å and about 500 Å.

(9) The method of (7) or (8), wherein the Amberlyst polymeric resin comprises Amberlyst 15.

(10) The method of (6), wherein the ion-exchange resin is a Nafion polymeric resin.

(11) The method of (10), wherein the Nafion polymeric resin comprises NR50, N115, N117, N324, N424, N1110, SAC-13, or a combination thereof.

(12) The method of (6), wherein the Lewis-acidic heterogeneous reagent is Al MCM-41, MCM-41, MCM-48, SBA-15, SBA-16, ZSM-5, ZSM-11, ZSM-22, ZSM-23, ZSM-35, SAPO-11, SAPO-34, SSZ-13, TS-1, KIT-5, KIT-6, FDU-12, Beta, X-type, Y-type, Linde type A, Linde type L, Linde type X, Linde type Y, Faujasite, Mordenite, Ferrierite, Montmorillonite K10, K30, KSF, Clayzic, bentonite, or a combination thereof.

(13) The method of (12), wherein the acidic heterogeneous reagent has a pore diameter of between about 0.1 nm and about 100 nm, a particle size of between about 0.1 μm and about 50 μm, a Si/Al ratio of between about 5 and about 1500, or a combination thereof.

(14) The method of (12) or (13), wherein the Lewis-acidic heterogeneous reagent is H-ZSM-5, with a Si/Al ratio of about 38, a pore size of about 5 Å, and a particle size of about 2 μm.

(15) The method of (12) or (13), wherein the Lewis-acidic heterogeneous reagent is Na-ZSM-5, with a Si/l ratio of about 38, a pore size of about 5 Å, and a particle size of about 2 μm.

(16) The method of (12) or (13), wherein the Lewis-acidic heterogeneous reagent is Al-MCM-41 with a Si/Al ratio of about 25, and a pore diameter of about 2.7 nm.

(17) The method of any one of (1) to (16), wherein the aprotic-solvent system comprises a class III solvent.

(18) The method of (17), wherein the class III solvent is heptane.

(19) The method of any one of (1) to (18), wherein prior to being converted to the composition comprising the Δ9-THC and the Δ8-THC, the CBD is dissolved in the aprotic-solvent system at a concentration between about 0.001 M and about 2 M.

(20) The method of any one of (1) to (19), wherein the threshold reaction temperature is between about 20° C. and about 100° C.

(21) The method of any one of (1) to (20), wherein the threshold reaction time is between about 10 minutes and about 72 hours.

(22) The method of any one of (1) to (21), wherein the Lewis-acidic heterogeneous reagent has a reagent loading between about 0.1 molar equivalents and about 100 molar equivalents relative to the CBD.

(23) The method of any one of (1) to (22), further comprising isolating the composition from the acidic heterogeneous reagent by a solid-liquid separation technique.

(24) The method of (23), wherein the solid-liquid separation technique comprises filtration, decantation, centrifugation, or a combination thereof.

(25) The method of any one of claims 1) to (24), wherein the CBD is a component of a distillate, an isolate, a concentrate, an extract, or a combination thereof.

(26) The method of (25), wherein the extract is a crude extract from hemp.

(27) The method of any one of (1) to (26), wherein the Δ9-THC:Δ8-THC ratio of the composition is greater than about 10.0:1.0.

(28) The method of any one of (1) to (26), wherein the Δ9-THC:Δ8-THC ratio of the composition is greater than about 100.0:1.0.

(29) The method of any one of (1) to (26), wherein the Δ9-THC:Δ8-THC ratio of the composition is greater than about 1000.0:1.0.

(30) A method for converting cannabidiol (CBD) into primarily Δ9-tetrahydrocannabinol (Δ9-THC), the method comprising contacting the CBD with a Lewis-acidic heterogeneous reagent under reaction conditions comprising: (i) an aprotic-solvent system; (ii) a reaction temperature that is less than a threshold reaction temperature for the Lewis-acidic heterogeneous reagent and the aprotic-solvent system; and (iii) a reaction time that is less than a threshold reaction time for the Lewis-acidic heterogeneous reagent, the aprotic-solvent system, and the reaction temperature.

(31) The method of (30), wherein the Lewis-acidic heterogeneous reagent is a Brønsted-acidic heterogeneous reagent.

(32) The method of (30) or (31), wherein the Lewis-acidic heterogeneous reagent has a Hammett-acidity value (Ho) of between about −8.0 and about 0.0.

(33) The method of any one of (30) to (32), wherein the Lewis-acidic heterogeneous reagent has a temperature-programmed desorption value of between about 7.5 and about 0.0 as determined with reference to ammonia (TPDNH3).

(34) The method of any one of (30) to (33), wherein the Lewis-acidic heterogeneous reagent has a heat of absorption value of between about −165 and about −100 as determined with reference to ammonia (ΔHoads NH3).

(35) The method of (30), wherein the Lewis-acidic heterogeneous reagent comprises an ion-exchange resin, a microporous silicate, a mesoporous silicate, a phyllosilicate, or a combination thereof.

(36) The method of (35), wherein the ion-exchange resin is an Amberlyst polymeric resin.

(37) The method of (36), wherein the Amberlyst polymeric resin has a surface area of between about 20 m2/g and about 80 m2/g and an average pore diameter of between about 100 Å and about 500 Å.

(38) The method of (36) or (37), wherein the Amberlyst polymeric resin comprises Amberlyst 15.

(39) The method of (35), wherein the ion-exchange resin is a Nafion polymeric resin.

(40) The method of (39), wherein the Nafion polymeric resin comprises NR50, N115, N117, N324, N424, N1110, SAC-13, or a combination thereof.

(41) The method of (35), wherein the Lewis-acidic heterogeneous reagent is Al MCM-41, MCM-41, MCM-48, SBA-15, SBA-16, ZSM-5, ZSM-11, ZSM-22, ZSM-23, ZSM-35, SAPO-11, SAPO-34, SSZ-13, TS-1, KIT-5, KIT-6, FDU-12, Beta, X-type, Y-type, Linde type A, Linde type L, Linde type X, Linde type Y, Faujasite, Mordenite, Ferrierite, Montmorillonite K10, K30, KSF, Clayzic, bentonite, or a combination thereof.

(42) The method of (41), wherein the acidic heterogeneous reagent has a pore diameter of between about 0.1 nm and about 100 nm, a particle size of between about 0.1 μm and about 50 μm, a Si/Al ratio of between about 5 and about 1500, or a combination thereof.

(43) The method of (41) or (42), wherein the Lewis-acidic heterogeneous reagent is H-ZSM-5, with a Si/Al ratio of about 38, a pore size of about 5 Å, and a particle size of about 2 μm.

(44) The method of (41) or (42), wherein the Lewis-acidic heterogeneous reagent is Na-ZSM-5, with a Si/Al ratio of about 38, a pore size of about 5 Å, and a particle size of about 2 μm.

(45) The method of (41) or (42), wherein the Lewis-acidic heterogeneous reagent is Al-MCM-41 with a Si/Al ratio of about 25, and a pore diameter of about 2.7 nm.

(46) The method of any one of (30) to (45), wherein the aprotic-solvent system comprises a class III solvent.

(47) The method of (46), wherein the class III solvent is heptane.

(48) The method of any one of (30) to (47), wherein prior to being converted to the Δ9-THC, the CBD is dissolved in the aprotic-solvent system at a concentration between about 0.001 M and about 2 M.

(49) The method of any one of (30) to (48), wherein the threshold reaction temperature is between about 20° C. and about 100° C.

(50) The method of any one of (30) to (49), wherein the threshold reaction time is between about 10 minutes and about 72 hours.

(51) The method of any one of (30) to (50), wherein the Lewis-acidic heterogeneous reagent has a reagent loading between about 0.1 molar equivalents and about 100 molar equivalents relative to the CBD.

(52) The method of any one of (30) to (51), further comprising isolating the composition from the acidic heterogeneous reagent by a solid-liquid separation technique.

(53) The method of (52), wherein the solid-liquid separation technique comprises filtration, decantation, centrifugation, or a combination thereof.

(54) The method of any one of (30) to (53), wherein the CBD is a component of a distillate, an isolate, a concentrate, an extract, or a combination thereof.

(55) The method of (54), wherein the extract is a crude extract from hemp.

(56) The method of any one of (30) to (55), wherein contacting the CBD with a Lewis-acidic heterogeneous reagent includes contacting the CBD with a Lewis-acidic heterogeneous reagent to produce a reaction product comprising primarily Δ9-THC, wherein the reaction product comprises at least 77% Δ9-THC.

(57) A method for converting cannabidiol (CBD) into a composition comprising Δ9-tetrahydrocannabinol (Δ9-THC) and Δ8-tetrahydrocannabinol (Δ8-THC), wherein the composition has a Δ9-THC:Δ8-THC ratio that is greater than 1.0:1.0, the method comprising contacting the CBD with a Lewis-acidic heterogeneous reagent under neat reaction conditions comprising: (i) a reaction temperature that is less than a threshold reaction temperature for the Lewis-acidic heterogeneous reagent; and (ii) a reaction time that is less than a threshold reaction time for the Lewis-acidic heterogeneous reagent and the reaction temperature.

(58) The method of (57), wherein the Lewis-acidic heterogeneous reagent is a Brønsted-acidic heterogeneous reagent.

(59) The method of (57) or (58), wherein the Lewis-acidic heterogeneous reagent has a Hammett-acidity value (Ho) of between about −8.0 and about 0.0.

(60) The method of any one of (57) to (59), wherein the Lewis-acidic heterogeneous reagent has a temperature-programmed desorption value of between about 7.5 and about 0.0 as determined with reference to ammonia (TPDNH3).

(61) The method of any one of (57) to (60), wherein the Lewis-acidic heterogeneous reagent has a heat of absorption value of between about −165 and about −100 as determined with reference to ammonia (ΔHoads NH3).

(62) The method of (57), wherein the Lewis-acidic heterogeneous reagent comprises an ion-exchange resin, a microporous silicate, a mesoporous silicate, a phyllosilicate, or a combination thereof.

(63) The method of (62), wherein the ion-exchange resin is an Amberlyst polymeric resin.

(64) The method of (63), wherein the Amberlyst polymeric resin has a surface area of between about 20 m2/g and about 80 m2/g and an average pore diameter of between about 100 Å and about 500 Å.

(65) The method of (63) or (64), wherein the Amberlyst polymeric resin comprises Amberlyst 15.

(66) The method of (62), wherein the ion-exchange resin is a Nafion polymeric resin.

(67) The method of (66), wherein the Nafion polymeric resin comprises NR50, N115, N117, N324, N424, N1110, SAC-13, or a combination thereof.

(68) The method of (62), wherein the Lewis-acidic heterogeneous reagent is Al MCM-41, MCM-41, MCM-48, SBA-15, SBA-16, ZSM-5, ZSM-11, ZSM-22, ZSM-23, ZSM-35, SAPO-11, SAPO-34, SSZ-13, TS-1, KIT-5, KIT-6, FDU-12, Beta, X-type, Y-type, Linde type A, Linde type L, Linde type X, Linde type Y, Faujasite, Mordenite, Ferrierite, Montmorillonite K10, K30, KSF, Clayzic, bentonite, or a combination thereof.

(69) The method of (68), wherein the acidic heterogeneous reagent has a pore diameter of between about 0.1 nm and about 100 nm, a particle size of between about 0.1 μm and about 50 μm, a Si/Al ratio of between about 5 and about 1500, or a combination thereof.

(70) The method of (68) or (69), wherein the Lewis-acidic heterogeneous reagent is H-ZSM-5, with a Si/Al ratio of about 38, a pore size of about 5 Å, and a particle size of about 2 μm.

(71) The method of (68) or (69), wherein the Lewis-acidic heterogeneous reagent is Na-ZSM-5, with a Si/Al ratio of about 38, a pore size of about 5 Å, and a particle size of about 2 μm.

(72) The method of (68) or (69), wherein the Lewis-acidic heterogeneous reagent is Al-MCM-41 with a Si/Al ratio of about 25, and a pore diameter of about 2.7 nm.

(73) The method of any one of (57) to (72), wherein the threshold reaction temperature is between about 20° C. and about 100° C.

(74) The method of any one of (57) to (73), wherein the threshold reaction time is between about 10 minutes and about 72 hours.

(75) The method of any one of (57) to (74), wherein the Lewis-acidic heterogeneous reagent has a reagent loading between about 0.1 molar equivalents and about 100 molar equivalents relative to the CBD.

(76) The method of any one of (57) to (75), wherein the CBD is a component of a distillate, an isolate, a concentrate, an extract, or a combination thereof.

(77) The method of (76), wherein the extract is a crude extract from hemp.

(78) The method of any one of (57) to (77), wherein the Δ9-THC:Δ8-THC ratio of the composition is greater than about 10.0:1.0.

(79) The method of any one of (57) to (77), wherein the Δ9-THC:Δ8-THC ratio of the composition is greater than about 100.0:1.0.

(80) The method of any one of (57) to (77), wherein the Δ9-THC:Δ8-THC ratio of the composition is greater than about 1000.0:1.0.

(81) A method for converting cannabidiol (CBD) into primarily Δ9-tetrahydrocannabinol (Δ9-THC), the method comprising contacting the CBD with a Lewis-acidic heterogeneous reagent under reaction conditions comprising: (i) a reaction temperature that is less than a threshold reaction temperature for the Lewis-acidic heterogeneous reagent; and (ii) a reaction time that is less than a threshold reaction time for the Lewis-acidic heterogeneous reagent and the reaction temperature.

(82) The method of (81), wherein the Lewis-acidic heterogeneous reagent is a Brønsted-acidic heterogeneous reagent.

(83) The method of (81) or (82), wherein the Lewis-acidic heterogeneous reagent has a Hammett-acidity value (Ho) of between about −8.0 and about 0.0.

(84) The method of any one of (81) to (83), wherein the Lewis-acidic heterogeneous reagent has a temperature-programmed desorption value of between about 7.5 and about 0.0 as determined with reference to ammonia (TPDNH3).

(85) The method of any one of (81) to (84), wherein the Lewis-acidic heterogeneous reagent has a heat of absorption value of between about −165 and about −100 as determined with reference to ammonia (ΔHoads NH3).

(86) The method of (81), wherein the Lewis-acidic heterogeneous reagent comprises an ion-exchange resin, a microporous silicate, a mesoporous silicate, a phyllosilicate, or a combination thereof.

(87) The method of (86), wherein the ion-exchange resin is an Amberlyst polymeric resin.

(88) The method of (87), wherein the Amberlyst polymeric resin has a surface area of between about 20 m2/g and about 80 m2/g and an average pore diameter of between about 100 Å and about 500 Å.

(89) The method of (87) or (88), wherein the Amberlyst polymeric resin comprises Amberlyst 15.

(90) The method of (86), wherein the ion-exchange resin is a Nafion polymeric resin.

(91) The method of (90), wherein the Nafion polymeric resin comprises NR50, N115, N117, N324, N424, N1110, SAC-13, or a combination thereof.

(92) The method of (86), wherein the Lewis-acidic heterogeneous reagent is Al MCM-41, MCM-41, MCM-48, SBA-15, SBA-16, ZSM-5, ZSM-11, ZSM-22, ZSM-23, ZSM-35, SAPO-11, SAPO-34, SSZ-13, TS-1, KIT-5, KIT-6, FDU-12, Beta, X-type, Y-type, Linde type A, Linde type L, Linde type X, Linde type Y, Faujasite, Mordenite, Ferrierite, Montmorillonite K10, K30, KSF, Clayzic, bentonite, or a combination thereof.

(93) The method of (92), wherein the acidic heterogeneous reagent has a pore diameter of between about 0.1 nm and about 100 nm, a particle size of between about 0.1 μm and about 50 μm, a Si/Al ratio of between about 5 and about 1500, or a combination thereof.

(94) The method of (92) or (93), wherein the Lewis-acidic heterogeneous reagent is H-ZSM-5, with a Si/Al ratio of about 38, a pore size of about 5 Å, and a particle size of about 2 μm.

(95) The method of (92) or (93), wherein the Lewis-acidic heterogeneous reagent is Na-ZSM-5, with a Si/Al ratio of about 38, a pore size of about 5 Å, and a particle size of about 2 μm.

(96) The method of (92) or (93), wherein the Lewis-acidic heterogeneous reagent is Al-MCM-41 with a Si/Al ratio of about 25, and a pore diameter of about 2.7 nm.

(97) The method of any one of (81) to (96), wherein the threshold reaction temperature is between about 20° C. and about 100° C.

(98) The method of any one of (81) to (97), wherein the threshold reaction time is between about 10 minutes and about 72 hours.

(99) The method of any one of (81) to (98), wherein the Lewis-acidic heterogeneous reagent has a reagent loading between about 0.1 molar equivalents and about 100 molar equivalents relative to the CBD.

(100) The method of any one of (81) to (99), wherein the CBD is a component of a distillate, an isolate, a concentrate, an extract, or a combination thereof.

(101) The method of (100), wherein the extract is a crude extract from hemp.

(102) The method of (81), wherein contacting the CBD with a Lewis-acidic heterogeneous reagent includes contacting the CBD with a Lewis-acidic heterogeneous reagent to produce a reaction product comprising primarily Δ9-THC, wherein the reaction product comprises at least 62.9% Δ9-THC.

### EXAMPLES

EXAMPLE 1 (E1l)—aprotic solvent: To a solution of CBD (500 mg, 1.59 mmol) in heptane (10 mL) was added ZSM-5 (1 g, ACS material, P-38, H⁺). The reaction was stirred at 60° C. for 18 hours. The reaction was cooled to room temperature and filtered using a fritted Buchner filtering funnel and then the reaction solvent was evaporated in vacuo. Analysis by HPLC showed Δ⁹-THC as the major product (see, TABLE 2).

COMPARISON EXAMPLE 1 (CE1)—aprotic solvent: To a solution of CBD (500 mg, 1.59 mmol) in heptane (10 mL) was added ZSM-5 (1 g, ACS material, P-38, H⁺). The reaction was stirred at 80° C. for 18 hours. The reaction was cooled to room temperature and filtered using a fritted Buchner filtering funnel and then the reaction solvent was evaporated in vacuo. Analysis by HPLC showed Δ⁸-THC as the major product (see, TABLE 2).

COMPARISON EXAMPLE 2 (CE2)—aprotic solvent/alternate Lewis-acidic heterogeneous reagent: To a solution of CBD (500 mg, 1.59 mmol) in heptane (10 mL) was added Amberlyst-15 (100 mg). The reaction was stirred at 60° C. for 2 hours. The reaction was cooled to room temperature and filtered using a fritted Buchner filtering funnel and then the reaction solvent was evaporated in vacuo. Analysis by HPLC showed Δ⁸-THC as the major product (see, TABLE 2).

EXAMPLE 2 (E2)—neat reaction conditions: A mixture of CBD (500 mg, 1.59 mmol) and ZSM-5 (1 g, ACS material, P-38, H⁺) was stirred at 100° C. for 30 minutes. The reaction was cooled to room temperature, diluted with 30 mL of TBME, and filtered using a fritted Buchner filtering funnel and then the reaction solvent was evaporated in vacuo. Analysis by HPLC showed Δ⁹-THC as the major product.

COMPARISON EXAMPLE 3 (CE3)—neat reaction conditions: A mixture of CBD (500 mg, 1.59 mmol) and ZSM-5 (1 g, ACS material, P-38, H⁺) was stirred at 100° C. for 18 hours. The reaction was cooled to room temperature, diluted with 30 mL of TBME, and filtered using a fritted Buchner filtering funnel and then the reaction solvent was evaporated in vacuo. Analysis by HPLC showed Δ⁸-THC as the major product.

TABLE 2
HPLC results from EXAMPLES E1, CE1, CE2, E2, and
CE3 (E = example; CE = comparison example).
Percentage values for CBD, Δ9-THC and Δ8-
THC were determined by HPLC-DAD (215 nm).

| | CBD | Δ⁹-THC | Δ⁸-THC | |
|---|---|---|---|---|
| Example | (%) | (%) | (%) | Δ⁹-THC:Δ⁸- THC |
| E1 | 50.8 | 32.6 | 10.5 | 3.1:1.0 |
| CE1 | 1.5 | 36.3 | 55.2 | 1.0:1.5 |
| CE2 | 0.1 | 5.3 | 81.1 | 1.0:15.3 |
| E2 | 46.8 | 22.8 | 13.7 | 1.7:1.0 |
| CE3 | 0.2 | 6.0 | 74.0 | 1.0:12.3 |

EXAMPLE 3: The effect of the silica to alumina ratio (molecular ratio, MR). in the ZSM-5 catalyst on the Δ⁹-THC:Δ⁸-THC ratio was studied.

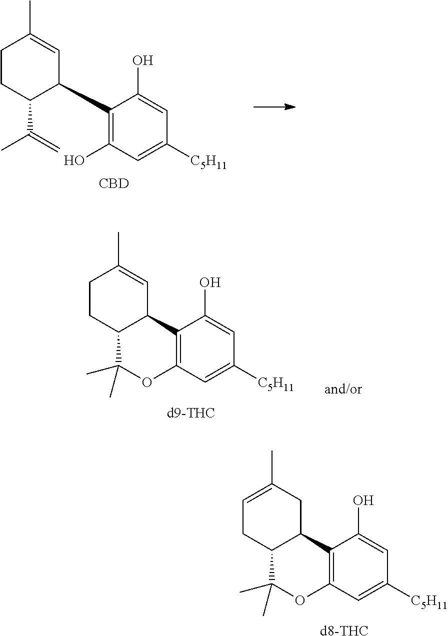

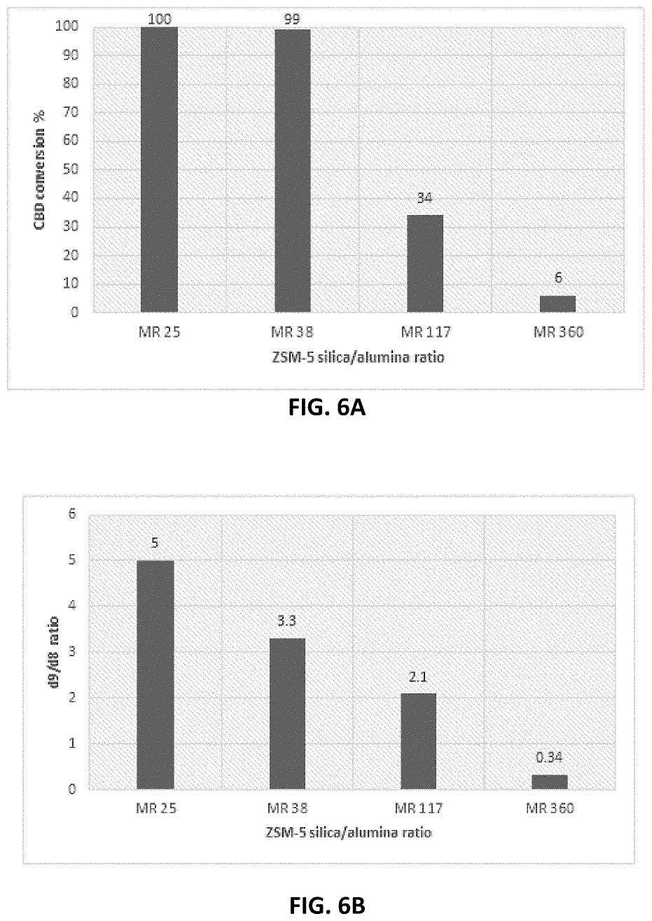

To a solution of CBD isolate (500 mg, 1.59 mmol) in heptane (10 mL) was added ZSM-5 (500 mg; 100% mass equivalent; MeQ). The reaction was stirred at 100° C. for 2 hours. The reaction was cooled to room temperature, filtered and the reaction solvent was evaporated in vacuo. As shown in FIG. 6, increasing the alumina sites provided a higher CBD conversion (FIG. 6A) but lower Δ⁹-THC:Δ⁸-THC ratio under the reaction conditions used (FIG. 6B).

EXAMPLE 4: A mixture of CBD isolate (500 mg, 1.59 mmol) and acidic alumina (100 mg; 100% MeQ) in heptane (10 mL) was stirred at 110° C. for 24 h. The reaction was cooled to room temperature, diluted with TBME, filtered and the reaction solvent was evaporated in vacuo. Analysis by HPLC showed Δ⁹-THC as the major product.

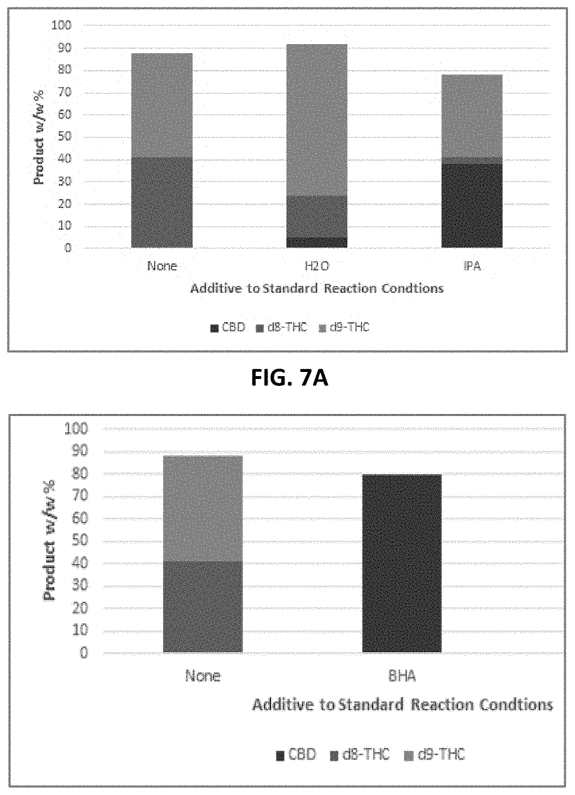

EXAMPLE 5: Experiments were performed to study the effect of additives on CBD conversion. The reactions were performed using the procedure described in Example 4, with the inclusion of an additive in the reaction mixture and a reaction temperature of 100° C. Water, isopropyl alcohol and butylated hydroxyanisole (BHA) were each studied as additives at 3 w/w %. As shown in FIG. 7A, the addition of water resulted in a small amount of CBD remaining while isopropyl alcohol moderately reduced conversion and BHA completed prevented the conversion under the reaction conditions tested (FIG. 7B).

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are dis-cussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1.-34. (canceled)

35. A method for converting cannabidiol (CBD) into Δ⁹-tetrahydrocannabinol (Δ⁹-THC), the method comprising contacting the CBD with a Lewis-acidic heterogeneous reagent in an aprotic-solvent system at a reaction temperature that is less than 65° C.

36. The method of claim 35, wherein the Δ⁹-THC is a component of a composition that further comprises Δ⁸-tetrahydrocannabinol (Δ⁸-THC), and wherein the composition has a Δ⁹-THC:Δ⁸-THC ratio that is greater than 1.0:1.0.

37. The method of claim 35, wherein the Lewis-acidic heterogeneous reagent is a microporous silicate.

38. The method of claim 37, wherein the microporous silicate is a zeolite which is ZSM-5, ZSM-11, ZSM-22, ZSM-23, ZSM-35, SAPO-11, SAPO-34, SSZ-13, TS-1, Beta, X-type, Y-type, Linde type A, Linde type L, Linde type X, Linde type Y, or any combination thereof.

39. The method of claim 37, wherein the microporous silicate is a zeolite which is ZSM-5.

40. The method of claim 35, wherein the aprotic-solvent system comprises dimethyl sulfoxide, ethyl acetate, dichloromethane, chloroform, toluene, pentane, heptane, hexane, diethyl ether, tert-butyl methyl ether, tetrahydrofuran, dioxane, dimethylformamide, dimethylacetamide, N-methylpyrrolidone, anisole, butyl acetate, cumene, ethyl formate, isobutyl acetate, isopropyl acetate, methyl acetate, methylethylketone, methylisobutylketone, propyl acetate, cyclohexane, para-xylene, meta-xylene, ortho-xylene, 1,2-dichloroethane, or any combination thereof.

41. The method of claim 35, wherein the aprotic-solvent system is heptane.

42. The method of claim 35, wherein the CBD is a component of a distillate, an isolate, a concentrate or an extract.

43. The method of claim 42, wherein the extract is a crude extract from hemp.

44. The method of claim 35, wherein the reaction temperature is about 60° C.

45. The method of claim 35, wherein the reaction temperature is room temperature.
